(19) United States
(12) Patent Application Publication
(10) Pub. No.: US 2016/0194369 A1
(43) Pub. Date: Jul. 7, 2016

(54) METHODS AND COMPOSITIONS FOR CO-EXPRESSION OF POLYPEPTIDES OF INEREST AND IL6

(71) Applicants: Janice CHEN, New York, NY (US); Shuangping SHI, Kenilworth, NJ (US)

(72) Inventors: Janice Chen, New York, NY (US); Shuangping Shi, Mountain Lakes, NJ (US)

(73) Assignee: Merck Sharp & Dohme Corp., Rahway, NJ (US)

(21) Appl. No.: 14/911,572

(22) PCT Filed: Aug. 7, 2014
(86) PCT No.: PCT/US14/50092

§ 371 Date: Feb. 11, 2016

Related U.S. Application Data
(60) Provisional application No. 61/864,809, filed on Aug. 12, 2013.

(51) Int. Cl. C07K 14/54

(52) U.S. Cl. CPC C07K 14/5412

(57) ABSTRACT

The present invention provides a CHO cell that includes a polynucleotide encoding a polypeptide of interest. The CHO cell may include a heterologous polynucleotide encoding IL6 and/or an IL6 pathway member or, in the absence of the heterologous IL6 or an IL6 pathway member polynucleotide, the CHO cell can be cultured in the presence of exogenously added IL6 polypeptide. Such CHO cells exhibit superior expression of the polypeptide of interest in culture. Methods for expressing a polypeptide of interest and methods for making such CHO cells are part of the present invention.

The present invention claims the benefit of U.S. Provisional Patent Application No. 61/864,809; filed Aug. 12, 2013; which is herein incorporated by reference in its entirety.

### FIELD OF THE INVENTION

The present invention relates to a Chinese hamster ovary (CHO) cell including a polynucleotide encoding a polypeptide of interest. In the presence of IL6, the CHO cells exhibit superior expression of the polypeptide of interest.

### BACKGROUND OF THE INVENTION

The generation of a stable, high productivity cell line is a key step in the manufacture of a therapeutic protein. An ideal production cell line will have a combination of fast growth and high specific-productivity, two features that are desirable for downstream scale-up and manufacturing processes. Moreover, a high-yield production cell lines are favorable because they require smaller production vessels and fewer production batches, allowing for reduced costs of goods for commercial supplies.

The cell line development process begins with the transfection of a vector carrying the gene of interest into host cells. Transfected cells are selected using a variety of systems, such as antibiotic resistance or metabolic selection markers, for several weeks. Because the level of recombinant protein expression varies from cell to cell, single cell subcloning is required to isolate cells exhibiting high titers. However, mammalian cells tend to suffer from poor cloning efficiency at extremely low cell density, resulting in low recovery after subcloning. For cells that do recover, several weeks are required for a single cell to expand into a colony large enough such that recombinant protein titers can be measured. Colonies exhibiting high titers are then harvested, expanded, and adapted to suspension in a process that can take another several weeks. Once in suspension, the productivity and stability of individual cell lines can be assessed. As each cell line exhibits different growth, productivity, and stability profiles, hundreds of clones are typically screened in a laborious process to identify a small number of cell lines with the most desirable characteristics. Thus, the cell line development process could benefit from a strategy to improve both the efficiency of the clone selection process and cell line productivity.

Interleukin-6 (IL6) is known to be an inflammatory cytokine produced by immune cells such as macrophages, dendritic cells, mast cells and B cells. Overexpression of IL6 is associated with several inflammatory diseases such as rheumatoid arthritis. A practitioner of ordinary skill in the art would not have expected IL6 to have a beneficial effect on the ability of CHO cells to express recombinant protein.

### SUMMARY OF THE INVENTION

The present invention provides an isolated Chinese hamster ovary (CHO) cell (e.g., that lacks a functional FUT8 polypeptide; and/or lacks a functional glutamine synthase polypeptide; and/or lacks a functional endogenous DHFR polypeptide) comprising (i) a heterologous polynucleotide encoding a polypeptide of interest (e.g., an immunoglobulin chain (heavy or light) of an antibody or antigen-binding fragment thereof) and (ii) a heterologous polynucleotide encoding IL6 polypeptide which, when cultured, the IL6 polypeptide (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) is expressed and secreted. Furthermore, the present invention provides an isolated Chinese hamster ovary (CHO) cell (e.g., that lacks a functional FUT8 polypeptide; and/or lacks a functional glutamine synthase polypeptide; and/or lacks a functional endogenous DHFR polypeptide) comprising a polynucleotide encoding a polypeptide of interest (e.g., an immunoglobulin chain (heavy or light) of an antibody or antigen-binding fragment thereof) which is in the presence of IL6 polypeptide (e.g., Chinese hamster, human, mouse or rat IL6), e.g., which has been exogenously added to the cells, e.g., to the culture medium of the cells, e.g., wherein such cells include or do not include an IL6 polynucleotide. The present invention also encompasses a composition comprising any of such CHO cells in a culture medium, buffer or carrier. In an embodiment of the invention, the cell expresses a constitutively active gp130 such as gp130ΔYY, e.g., wherein the cell is heterozygous or homozygous for the gp130ΔYY allele.

The present invention provides a method for increasing the quantity of protein of interest expressed from a CHO cell having a heterologous polynucleotide encoding the protein of interest; or for increasing the rate of growth of a CHO cell having a heterologous polynucleotide encoding the protein of interest; comprising co-expressing IL6 (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) with the polypeptide of interest in the CHO cell (e.g., while the cell is being cultured); or exposing a CHO cell having a polynucleotide encoding the protein of interest to exogenous IL6 polypeptide (e.g., while the cell is being cultured).

The present invention also provides a method for increasing the number of CHO cells that survive transfection with a polynucleotide (e.g., a polynucleotide of interest) comprising transfecting the cells with a polynucleotide encoding IL6 (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) and/or by exposing the cells transfected with the polynucleotide to exogenous IL6 polypeptide.

The present invention also provides a method for making a IL6⁺-CHO cell of the present invention comprising introducing a polynucleotide encoding a polypeptide of interest and a polynucleotide encoding IL6 polypeptide (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) into a Chinese hamster ovary cell. Any cell produced by such a method (as well as methods of use and methods of making the same; as discussed herein) is within the scope of the present invention.

The present invention also provides a method for making a polypeptide of interest comprising culturing a IL6⁺-CHO cell under conditions wherein the polypeptide of interest and the IL6 polypeptide (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) are expressed. In an embodiment of the invention, the method comprises the steps of: (a) introducing a polynucleotide encoding a polypeptide of interest and a polynucleotide encoding IL6 polypeptide into a CHO cell; and (b) culturing the cell under conditions wherein the polypeptide of interest and the IL6 polypeptide are expressed. Optionally, the polypeptide of interest is purified away from the cell, cell components, culture medium and/or IL6 polypeptide.

The present invention also provides a method for making a polypeptide of interest comprising the steps of: (a) introducing a polynucleotide encoding a polypeptide of interest into a CHO cell; (b) introducing the cell into a growth medium; (c) adding exogenous IL6 polypeptide (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) to the growth medium; and (b) culturing the cell under conditions wherein the polypeptide of interest is expressed. Optionally, the polypeptide of interest is purified away from the cell, cell components, culture medium and/or IL6 polypeptide.

### BRIEF DESCRIPTION OF THE FIGURES

FIG. 1: Effect of exogenous IL6 increases on the percentage of canP-mIgG^{hi} cells at the pool level. CHOK1SV cells were transfected with pCanP-mIgG and then selected based on the selectable marker in the transfected vector backbone for two weeks in the presence or absence of 100 ng/ml human IL6. The pool of transfected cells was harvested and stained with a phycoerythrin-conjugated antibody against mouse IgG to measure canP-mIgG expression. (A) Dot plot showing forward scatter on the X-axis and PE staining on the Y-axis. Numbers show percentage of high PE+ cells. (B) Histogram showing PE staining on the X-axis and cell counts on the Y-axis. Numbers show mean fluorescence intensity (MFI) of PE+ cells.

FIG. 2: Effect of exogenous IL6 on canP-mIgG titers at the pool and 96-well stages. Transfected CHOK1SV cells expressing the highest levels of canP-mIgG were subcloned by fluorescence-activated cell sorting (FACS) into five 96-well Plates with or without 100 ng/ml human IL6. Cells were allowed to recover for 14 days, then wells containing recovered colonies were sampled and assayed with an mIgG ELISA. (A) Time course of canP-mIgG titers in the transfected pool prior to subcloning. (B) canP-mIgG titers of individual clones in 96-well plates two weeks post-subcloning.

FIG. 3: Effect of co-expressed Chinese hamster or human IL6 on cell recovery and rate of colony emergence following subcloning. CHOK1SV cells were transfected with pCanP-mIgG, pCanP-mIgG+hIL6, or pCanP-mIgG+CHOIL6. After 14 days of selection based on the transfected vector selectable marker, cells were either subcloned into 96-well plates (A) based on staining with Dylight 488-conjugated Protein A or (B) randomly, based on forward and side scatter. Upper panels show number of colonies that emerged 7, 10, and 14 days post-subcloning. Lower panels show percent recovery of clones at 7, 10, and 14 days post-subcloning.

FIG. 4: Effect of Chinese hamster or human IL6 on cell recovery and rate of colony emergence following subcloning. Images of colonies on select 96-well plates 14 days post-subcloning. Upper panels show clones that were sorted based on Protein A staining. Lower panels show clones that were randomly sorted based on forward and side scatter.

FIG. 5: Effect of Chinese hamster or human IL6 co-expression on canP-mIgG titers at the 96-well stage. canP-mIgG titers of individual clones were assayed by mIgG ELISA two weeks post-subcloning. (A) shows clones that were sorted based on Protein A staining. (B) shows clones that were randomly sorted based on forward and side scatter.

FIG. 6: Effect of Chinese hamster or human IL6 co-expression on canP-mIgG titers in fed batch mode. Clones with the highest canP-mIgG titers from each transfection were harvested from 96-well plates, adapted to suspension, and their productivity in fed batch was assessed. Titers were determined by Protein A-HPLC of crude, cell-free culture supernatants. n=7-9 clones per sort condition per transfection. Day 14 titers are shown for (A) clones that were sorted based on Protein A staining and (B) clones that were randomly sorted based on forward and side scatter. The bar and adjacent numeral denote mean canP-mIgG titer of clones for a given transfection.

FIG. 7: Co-expression of CHO IL-6 improved colony growth. Colony size of cells expressing the anti-PD1 antibody MK3475±Chinese hamster IL6 after 10 or 14 days of growth.

FIG. 8: Co-expression of CHO IL-6 increases titers and % positive clones at the 96-well stage.

FIG. 9: IL6 over-expression improved day 14 median titer.

FIG. 10: Higher median specific productivity observed when over-expressing IL6.

FIG. 11: Gain-of-function mutations of gp130. Examples of several gp130 gain-of-function mutations which may be expressed in the cells of the present invention.

FIG. 12: gp130 constructs expressing human gp130ΔYY, gp130ΔFY or wild-type gp130 and canine TSLP-IgG2a.

FIG. 13: hGP130 mutant improved the fed batch 14 titer the most.

### DETAILED DESCRIPTION OF THE INVENTION

Chinese hamster ovary (CHO) cells are one of the most commonly used mammalian cell lines for the manufacture of complex biopharmaceuticals due to their robust growth in suspension culture, high yield of protein products, post-translational processing, and safety profile. The present invention provides a method for promoting cell growth and recombinant protein productivity of CHO cells by supplementing or co-expressing IL6 during the cell line development process. CHO cells transfected with a gene of interest and then selected and subcloned in the presence of IL6 showed enhanced cell recovery and accelerated colony emergence following subcloning. Moreover; colonies exhibited increased titers following subcloning. After the top clones were harvested and adapted to suspension, their productivity in a 14-day fed batch process was assessed. On average, clones co-expressing CHO IL6 produced 3.3-4.5-fold higher levels of the protein of interest than clones expressing the protein of interest alone. Thus, IL6 can be applied to the cell line development process to reduce clone selection timelines, improve cell line productivity, and facilitate the identification of highly-productive clones.

In addition, overexpression of IL6 pathway components in CHO cells (above that of a wild-type CHO cell) mimics the effect of IL6 co-expression without the need for a soluble ligand. For instance, overexpression of a constitutively active form of gp130 (e.g., gp130ΔYY) activates IL6 signaling pathways without the need for either exogenously supplemented or endogenously produced IL6. Gain-of-function mutations in gp130 lead to STAT3 activation and expression of IL6 target genes in the absence of IL6. Such mutations include those which are in-frame deletions that disrupt the gp130-IL6 interface and lead to ligand-independent activation. Overexpression of these mutant gp130s, as well as IL6 pathway components, e.g., downstream of gp130 (such as the transcription factors STAT3 or NF-IL6), mimic the effect of IL6 on CHO cells. A major advantage of these alternative approaches is not having to purify the therapeutic protein of interest from co-expressed IL6, which could be a significant component of the culture supernatant.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (DN. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, at al. (eds.), Current Protocols in Molecular Biology, John Wiley & Sons, Inc. (1994).

A “polynucleotide”, “nucleic acid” or “nucleic acid molecule” DNA and RNA (e.g., mRNA), single or double stranded.

An “endogenous” polynucleotide or polypeptide is present normally in a wild-type host cell such as a CHO cell.

A “heterologous” polynucleotide or “heterologous” polypeptide is not present normally in a wild-type host cell such as a CHO cells but was introduced to the cell.

Substances that are “exogenous” are added to a composition, such as IL6 polypeptide which is added to a culture medium.

A “nucleotide sequence” is a series of nucleotide bases (also called “nucleotides”) in a nucleic acid, such as DNA or RNA, and means any chain of two or more nucleotides.

An “amino acid sequence” refers to a series of two or more amino acids in a protein, peptide or polypeptide.

A “protein”, “peptide” or “polypeptide” includes a contiguous string of two or more amino acids.

The terms “isolated polynucleotide” or “isolated polypeptide” include a polynucleotide (e.g., RNA or DNA molecule, or a mixed polymer) or a polypeptide, respectively, which are partially (to any degree) or fully separated from other components that are normally found in cells or in recombinant DNA expression systems. These components include, but are not limited to, cell membranes, cell walls, ribosomes, polymerases, serum components and extraneous genomic sequences.

The term “host cell” includes any cell of any organism (e.g., a CHO cell) that is selected, modified, transfected, transformed, grown, or used or manipulated in any way, for the production of a substance by the cell, for example the expression or replication, by the cell, of a gene, a DNA or RNA sequence or a protein.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

A coding sequence is “operably linked to” transcriptional and translational control sequences in a cell when the sequences direct RNA polymerase mediated transcription of the coding sequence into RNA, preferably mRNA, which then may be RNA spliced (if it contains introns) and, optionally, translated into a protein encoded by the coding sequence. IL6 and/or polynucleotides of interest, in CHO cells of the present invention (and methods of use thereof, as discussed herein), may, in some embodiments of the invention, be operably linked to transcriptional and/or translational control sequences.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “vector” includes a vehicle (e.g., a plasmid) by which a DNA or RNA sequence can be introduced into a host cell, so as to transform the host and, optionally, promote expression and/or replication of the introduced sequence.

Vectors that can be used in this invention include plasmids, viruses, bacteriophage, integratable DNA fragments, and other vehicles that may facilitate introduction of the nucleic acids into the genome of the host. Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al., (eds.), Vectors: A Survey of Molecular Cloning Vectors and Their Uses, 1988, Buttersworth, Boston, Mass.

The polynucleotides encoding polypeptides of interest and/or IL6 which are in the CHO cells of the present invention, may, in an embodiment of the invention, include one more selectable markers. Selectable markers include, for example, dihydrofolate reductase (DHFR), glutamine synthetase hygromycin-resistance, puromycin-resistance, or neomycin-resistance.

The term “transfection” or “transformation” refers to the process of introducing a polynucleotide into a cell (e.g., a CHO cell); e.g., by the electroporation or calcium phosphate method.

A cell that is “cultured” is grown in a culture medium (e.g., liquid or solid culture medium) under conditions favorable to such growth and, when the cell is transformed with a polynucleotide of interest and/or a polynucleotide encoding IL6 and/or an IL6 pathway member (e.g., gp130ΔYY), under conditions favorable to expression of the polypeptide of interest and/or IL6 polypeptide and/or an IL6 pathway member.

### Interleukin-6

The present invention provides CHO cells comprising one or more polynucleotides of interest (e.g., in a vector such as a plasmid, ectopic or chromosomally integrated) and one or more IL6 polynucleotides (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta e.g., in a vector such as a plasmid, ectopic or chromosomally integrated) and methods of use thereof (e.g., as discussed herein). The IL6 polynucleotide can encode full length IL6 or a functional fragment thereof or a functional mutational variant thereof, wherein the IL6 polypeptide is secreted when expressed and binds IL6R on the surface of a CHO cell.

The scope of the present invention includes embodiments wherein members of the IL6 pathway are modulated in the CHO cells, either in addition to direct exposure to IL6 protein or in place of direct exposure to IL6 protein. For example, the present invention includes embodiments wherein JAK tyrosine kinases of the CHO cells (e.g., JAK1, JAK2, and/or TYK2), which are associated with the cytoplasmic domain of gp130 and are trans-phosphorylated in response to IL6 stimulation, are up-regulated e.g., by increasing their expression (e.g., by transforming CHO cells with a heterologous polynucleotide encoding a JAK which is expressed in the cell) and/or by exposure to activating substances (e.g., small molecule agonists and/or antagonists of JAK antagonists). Activated JAKs phosphorylate specific tyrosine residues in the cytoplasmic domain of gp130, which serve as docking sites for the latent STAT transcription factors. Upon recruitment to the phosphotyrosine motifs of gp130, the STAT proteins are then phosphorylated by JAKs. Phosphorylated STATs form homo- and heterodimers and translocate to the nucleus, where they activate transcription of their target genes. STAT3 is predominantly activated downstream of IL6, and to a lesser extent, STAT1. Thus, the present invention also includes embodiments wherein STATs (e.g., STAT1 and/or STAT3) are up-regulated, either by increasing their expression (e.g., by transforming CHO cells with a heterologous polynucleotide encoding a STAT which is expressed in the cell) or by inducing their phosphorylation (e.g., by exposure to a kinase such as a JAK).

Another integral pathway activated downstream of IL6 is the MAPK pathway. Upon IL6 stimulation, the tyrosine phosphatase SHP2 is recruited to gp130 and phosphorylated by JAKs. SHP2 acts as an adaptor protein linking activation of the Ras pathway to the downstream ERK, p38, and JNK MAPKs. A major mechanism by which MAPKs activate transcription factors is by post-translational modification, such as phosphorylation. Thus, the present invention includes embodiments wherein MAPK pathway members, e.g., SHP2, are up-regulated, e.g., by increasing their expression (e.g., by transforming CHO cells with a heterologous polynucleotide encoding SHP2 which is expressed in the cell) and/or by exposure to activating substances (e.g., small molecule agonists and/or antagonists of SHP2 antagonists). The present invention also includes embodiments wherein nuclear factor IL6 (NF-IL6), AP-1 and/or ETS family members are up-regulated, e.g., by increasing their expression (e.g., by transforming CHO cells with a heterologous polynucleotide encoding NF-IL6, AP-1 and/or ETS family members which is expressed in the cell) and/or by exposure of NF-IL6, AP-1 and/or ETS family members to MAPK (e.g., by transforming CHO cells with a heterologous polynucleotide encoding a MAPK which is expressed in the cell). NF-IL6, AP-1 and/or ETS family members are transcription factors activated downstream of MAPKs.

IL6 pathway members include any of IL6R, gp130 (e.g., the gp130ΔYY mutant, gp130ΔFY mutant, e.g., Chinese hamster or human), JAK1, JAK2, TYK2, STAT1, STAT3, SHP2, NF-IL6, AP-1 and an ETS family member (e.g., ETS, YAN, ELG, PEA3, ERF and ternary complex factor (TCF, e.g., ELK1, SAP1A, SAP1B)), e.g., from a mammal such as from human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta.

A CHO cell comprising an IL6 polynucleotide and/or IL6 pathway member (e.g., gp130ΔYY) polynucleotide; and, a polynucleotide of interest may be referred to herein as an “IL6⁺-CHO” cell. In an IL6⁺-CHO cell an IL6 polynucleotide or IL6 pathway member polynucleotide may be referred to herein as “heterologous”, and is (i) a polynucleotide that encodes IL6 or the pathway member which differs from IL6 or the pathway member from Chinese hamster at one or more nucleotides (e.g., which is human IL6, e.g., any of SEQ ID NOs: 2 and 4-8); or (ii) a polynucleotide encoding IL6 or the pathway member that is identical to Chinese hamster IL6 (e.g., SEQ ID NO: 3), wherein the polynucleotide has been added to a CHO cell and constitutes an additional cellular copy of the Chinese hamster IL6 or pathway member.

The present invention provides CHO cells comprising a heterologous IL6 and/or IL6 pathway member (e.g., gp130ΔYY) from any species having an endogenous IL6 gene and/or IL6 pathway (e.g., human or Chinese hamster IL6), optionally, operably linked to a promoter that drives expression of the IL6 and/or pathway member when the cell is cultured. IL6 polynucleotides and/or IL6 pathway member polynucleotides may, in an embodiment of the invention, be operably linked to a promoter that causes transcription in a CHO cell; such as a CMV promoter (e.g., immediate-early cytomegalovirus virus promoter), EF-1alpha promoter, Ubc promoter (human ubiquitin C promoter), SV40 promoter (simian virus 40 promoter) or the PGK promoter (murine phosphoglycerate kinase-1 promoter).

In an embodiment of the invention, human IL6 polypeptide comprises the amino acid sequence:

(SEQ ID NO: 2)
mnsfstsafg pvafslglll vlpaafpapv ppgedskdva
aphrqpltss eridkqiryi ldgisalrke tcnksnmces
skealaennl nlpkmaekdg cfqsgfneet clvkiitgll
efevyleylq nrfesseeqa ravqmstkvl iqflqkkakn
ldaittpdpt tnaslltklq aqnqwlqdmt thlilrsfke
flqsslralr qm

In an embodiment of the invention, IL6 polynucleotide encodes human IL6 polypeptide, e.g., SEQ ID NO: 2, for example, comprising the nucleotide sequence:

(SEQ ID NO: 1)
atgaactcct tctccacaag cgccttcggt ccagttgcct
tctccctggg gctgctcctg gtgttgcctg ctgccttccc
tgccccagta cccccaggag aagattccaa agatgtagcc
gccccacaca gacagccact cacctcttca gaacgaattg
acaaacaaat tcggtacatc ctcgacggca tctcagccct
gagaaaggag acatgtaaca agagtaacat gtgtgaaagc
agcaaagagg cactggcaga aaacaacctg aaccttccaa
agatggctga aaaagatgga tgcttccaat ctggattcaa
tgaggagact tgcctggtga aaatcatcac tggtcttttg
gagtttgagg tatacctaga gtacctccag aacagatttg
agagtagtga ggaacaagcc agagctgtgc agatgagtac
aaaagtcctg atccagttcc tgcagaaaaa ggcaaagaat
ctagatgcaa taaccacccc tgacccaacc acaaatgcca
gcctgctgac gaagctgcag gcacagaacc agtggctgca
ggacatgaca actcatctca ttctgcgcag ctttaaggag
ttcctgcagt ccagcctgag ggctcttcgg caaatgtag

In an embodiment of the invention, Chinese hamster (Cricetulus griseus) IL6 polypeptide comprises the amino acid sequence:

(SEQ ID NO: 3)
mkflsardfh plaflglmla vatalptsqv rrgdftedtt
pnrpvyttsq qvgglvthvl reifelrkel cnnnpdcmny
ddallennle lpviqrndgc yqtgynweic llkitsglld
yqiylefvtn nvqdnkkdka rvigsttktl sqifkqevkd
pdkivmpspt skailiekle sqkqwprtkt ielilkalee
flkvtmrstr qn

In an embodiment of the invention, Rattus norvegicus IL6 polypeptide comprises the amino acid sequence:

(SEQ ID NO: 4)
mkflsardfq pvaflglmll tatafptsqv rrgdftedtt
hnrpvyttsq vgglityvlr eilemrkelc ngnsdcmnsd
dalsennlkl peiqrndgcf qtgynqeicl lkicsgllef
rfylefvknn lqdnkkdkar viqsntetiv hifkqeikds
ykivlptpts nallmekles qkewlrtkti qlilkaleef
lkvtmrstrq t

In an embodiment of the invention, Mus musculus IL6 polypeptide comprises the amino acid sequence:

(SEQ ID NO: 5)
mlvtttafpt sqvrrgdfte dtthnrpvyt tsqvgglith
vlweivemrk elcngnsdcm nnddalaenn lklpeiqrnd
gcyqtgynqe icllkissgl leyhsyleym knnlkdnkkd
karvlqrdte tlihifnqev kdlhkivlpt pisnalltdk
lesqkewlrt ktiqfilksl eeflkvtlrs trqt

In an embodiment of the invention, Pan troglodytes IL6 polypeptide comprises the amino acid sequence:

(SEQ ID NO: 6)
mnsystsafg pvafslglll vlpaafpapv ppgedskdva
aphrqpltss eridkqiryi ldgisalrke tcnksnmces
skealaennl nlpkmaekdg cfqsgfneet clvkiitgll
efevyleylq nrfesseeqa ravqmstkvl iqflqkkakn
ldaittpdpt tnaslltklq aqnqwlqdmt thlilrsfke
flqsslralr qm

In an embodiment of the invention, Gorilla gorilla gorilla IL6 polypeptide comprises the amino acid sequence:

(SEQ ID NO: 7)
mnsystsafg pvafslglll vlpaafpapv ppgedskdva
aphrqpltss eridkqiryi ldgisalrke tcnksnmces
skealaennl nlpkmaekdg cfqsgfneet clvkiitgll
efevyleylq nrfesseeqa ravqmstkvl iqflqkkvgn
ldvittpdpt tnaslltklq aqnqwlqdmt thlilrsfke
flqsslralr qm

In an embodiment of the invention, Macaca mulatta IL6 polypeptide comprises the amino acid sequence:

(SEQ ID NO: 8)
mnsystsafg pvafslglll vlpaafpapv lpgedskdva
aphsqpltss eridkhiryi ldgisalrke tcnrsnmces
skealaennl nlpkmaekdg cfqsgfnedt clvkiitgll
efevyleylq nrfesseeqa ravqmstkvl iqflqkkakn
ldaittpept tnaslltklq aqnqwlqdmt thlilrsfke
flqsslralr qm

In an embodiment of the invention, the IL6 is any of those set forth below:

1. interleukin-6
[Ailuropoda
melanoleuca]
211 aa protein
AEY70473.1
GI: 373431041
5. PREDICTED:
interleukin-6
[Anas platyrhynchos]
177 aa protein
XP_005020599.1
GI: 514750321
6. interleukin-6
[Anser anser]
234 aa protein
AEB26769.1
GI: 328672205
8. interleukin-6
[Aotus lemurinus]
209 aa protein
AAF21298.1
GI: 6648940
9. interleukin-6
[Aotus nancymaae]
209 aa protein
AAD01536.1
GI: 4102682
10. interleukin-6
[Aotus nigriceps]
175 aa protein
AAF21297.1
GI: 6648938
11. interleukin-6
[Aotus vociferans]
160 aa protein
AAD01531.1
GI: 4102672
12. interleukin-6
[Bos grunniens]
46 aa protein
AAT02420.1
GI: 46849998
13. Interleukin-6,
partial [Bos
grunniens mutus]
181 aa protein
ELR52426.1
GI: 440901495
15. interleukin-6
precursor
[Bos taurus]
208 aa protein
NP_776348.1
GI: 27806867
17. Interleukin-6
[Bubalus
bubalus x
Bubalus
carabanensis]
208 aa protein
BAE76014.1
GI: 84993700
18. interleukin 6
[Callithrix jacchus]
212 aa protein
ABG54455.1
GI: 110180456
19. interleukin-6
[Camelus
dromedarius]
211 aa protein
ADH51721.1
GI: 296803305
24. interleukin-6
precursor
[Canis lupus
familiaris]
207 aa protein
NP_001003301.1
GI: 54607208
27. interleukin-6
[Capra hircus]
208 aa protein
ADP09351.1
GI: 310751797
28. PREDICTED:
interleukin-6
[Ceratotherium
simum simum]
214 aa protein
XP_004418987.1
GI: 478489215
29. interleukin 6
[Cercocebus
torquatus]
212 aa protein
AAA99972.1
GI: 514342
30. interleukin-6
[Cervus elaphus]
191 aa protein
AAS73282.1
GI: 45645330
31. Interleukin-6
[Chelonia mydas]
214 aa protein
EMP29850.1
GI: 465962690
32. interleukin-6
[Chlorocebus
sabaeus]
212 aa protein
ACI28913.1
GI: 208436712
33. Interleukin-6,
partial
[Columba livia]
154 aa protein
EMC81620.1
GI: 449270984
34. PREDICTED:
interleukin-6
[Condylura cristata]
211 aa protein
XP_004676819.1
GI: 507931152
36. PREDICTED:
interleukin-6
[Cricetulus griseus]
212 aa protein
XP_003507489.1
GI: 354490689
38. interleukin 6
(interferon, beta 2)
precursor
[Danio rerio]
231 aa protein
NP_001248378.1
GI: 387598083
39. PREDICTED:
interleukin-6
[Dasypus
novemcinctus]
214 aa protein
XP_004461419.1
GI: 488539604
40. interleukin-6
[Dicentrarchus
labrax]
143 aa protein
CAM32185.1
GI: 151175283
41. PREDICTED:
interleukin-6
[Echinops telfairi]
211 aa protein
XP_004702898.1
GI: 507646557
42. interleukin-6
[Epinephelus
coioides]
223 aa protein
AFE62919.1
GI: 380778719
46. interleukin-6
precursor
[Equus caballus]
208 aa protein
NP_001075965.1
GI: 126723700
50. interleukin-6
precursor
[Felis catus]
207 aa protein
NP_001009211.1
GI: 57163747
1236. interleukin 6
[Ailuropoda
melanoleuca]
211 aa protein
ABQ95347.1
GI: 148575269
1307. PREDICTED:
interleukin-6
[Anas platyrhynchos]
177 aa protein
XP_005020599.1
GI: 514750321
1322. interleukin-6
[Anser anser]
234 aa protein
AEB26769.1
GI: 328672205
1325. interleukin-6
[Aotus lemurinus]
209 aa protein
AAF21298.1
GI: 6648940
1326. interleukin-6
[Aotus nancymaae]
209 aa protein
AAD01536.1
GI: 4102682
1327. interleukin-6
[Aotus nigriceps]
175 aa protein
AAF21297.1
GI: 6648938
1328. interleukin-6
[Aotus vociferans]
160 aa protein
AAD01531.1
GI: 4102672
1339. Interleukin-6,
partial [Bos
grunniens mutus]
181 aa protein
ELR52426.1
GI: 440901495
1368. interleukin-6
precursor [Bos
taurus]
208 aa protein
NP_776348.1
GI: 27806867
1437. interleukin 6
[Bubalus bubalis]
208 aa protein
AAQ54301.1
GI: 33771329
1449. Interleukin-6
[Bubalus bubalis x
Bubalus
carabanensis]
208 aa protein
BAE76014.1
GI: 84993700
1450. Interleukin-6
[Bubalus
carabanensis]
208 aa protein
BAE76006.1
GI: 84993684
1453. interleukin-6
[Callithrix jacchus]
212 aa protein
ABG54455.1
GI: 110180456
1477. interleukin-6
precursor
[Callithrix jacchus]
212 aa protein
NP_001254700.1
GI: 391853094
1480. interleukin 6
[Camelus bactrianus]
211 aa protein
BAC75393.1
GI: 29603623
1482. interleukin-6
[Camelus
dromedarius]
211 aa protein
ADH51721.1
GI: 296803305
1504. interleukin-6
precursor [Canis
lupus familiaris]
207 aa protein
NP_001003301.1
GI: 54607208
1525. IL-6 precursor
[Capra hircus]
208 aa protein
BAA13118.1
GI: 1480068
1559. PREDICTED:
interleukin-6
[Ceratotherium
simum simum]
214 aa protein
XP_004418987.1
GI: 478489215
1579. interleukin 6
[Cercocebus
torquatus]
212 aa protein
AAA99972.1
GI: 514342
1580. interleukin-6
[Cervus elaphus]
191 aa protein
AAS73282.1
GI: 45645330
1584. Interleukin-6
[Chelonia mydas]
214 aa protein
EMP29850.1
GI: 465962690
1591. interleukin-6
[Chlorocebus
sabaeus]
212 aa protein
ACI28913.1
GI: 208436712
1603. Interleukin-6,
partial
[Columba livia]
154 aa protein
EMC81620.1
GI: 449270984
1613. PREDICTED:
interleukin-6
[Condylura cristata]
211 aa protein
XP_004676819.1
GI: 507931152
1630. interleukin 6,
partial [Coturnix
japonica]
146 aa protein
BAL02990.1
GI: 349500558
1654. PREDICTED:
interleukin-6
[Cricetulus griseus]
212 aa protein
XP_003507489.1
GI: 354490689
1682. interleukin-6a
[Cyprinus carpio]
232 aa protein
AGR82312.1
GI: 526260858
1708. interleukin 6
(interferon, beta 2)
precursor
[Danio rerio]
231 aa protein
NP_001248378.1
GI: 387598083
1715. PREDICTED:
interleukin-6
[Dasypus
novemcinctus]
214 aa protein
XP_004461419.1
GI: 488539604
1726. interleukin 6
precursor
[Delphinapterus
leucas]
208 aa protein
AAD42929.1
GI: 5381297
1731. interleukin-6
[Dicentrarchus
labrax]
143 aa protein
CAM32185.1
GI: 151175283
1736. PREDICTED:
interleukin-6
[Echinops telfairi]
211 aa protein
XP_004702898.1
GI: 507646557
1747. interleukin 6
[Enhydra lutris]
207 aa protein
AAB01428.1
GI: 1146344
1750. interleukin-6
[Epinephelus
coioides]
223 aa protein
AFE62919.1
GI: 380778719
1762. interleukin-6
precursor
[Equus caballus]
208 aa protein
NP_001075965.1
GI: 126723700
1797. interleukin-6
precursor
[Felis catus]
207 aa protein
NP_001009211.1
GI: 57163747
1819. PREDICTED:
interleukin-6
[Ficedula
albicollis]
281 aa protein
XP_005041194.1
GI: 524986498
1835. interleukin-6,
partial [Gadus
morhua]
169 aa protein
AEB96257.1
GI: 329668262
1852. interleukin-6
precursor
[Gallus gallus]
241 aa protein
NP_989959.1
GI: 45382889
1905. PREDICTED:
interleukin-6
[Gorilla gorilla
gorilla]
212 aa protein
XP_004045208.1
GI: 426355614
1925. Interleukin-6
[Heterocephalus
glaber]
218 aa protein
EHB06510.1
GI: 351703591
1989. interleukin-6
[Hippoglossus
hippoglossus]
226 aa protein
ADP55202.1
GI: 310975753
2121. interleukin-6
precursor
[Homo sapiens]
212 aa protein
NP_000591.1
GI: 10834984
2499. interleukin-6
[Human
herpesvirus 8]
204 aa protein
AFQ99135.1
GI: 402797604
2510. PREDICTED:
interleukin-6
[Jaculus jaculus]
333 aa protein
XP_004652710.1
GI: 507537371
2528. interleukin 6
[Lama glama]
211 aa protein
BAC75384.1
GI: 29603605
2530. interleukin 6
[Lepus californicus]
118 aa protein
AAF86663.1
GI: 9294773
2531. interleukin 6
[Lepus townsendii]
118 aa protein
AAF86664.1
GI: 9294775
2533. PREDICTED:
interleukin-6-like
[Loxodonta africana]
212 aa protein
XP_003407131.1
GI: 344270598
2550. interleukin-6
precursor [Macaca
fascicularis]
212 aa protein
BAA19148.1
GI: 1799528
2620. interleukin-6
precursor
[Macaca mulatta]
212 aa protein
NP_001036198.2
GI: 418203918
2622. interleukin 6
precursor
[Macaca thibetana]
212 aa protein
AAW33962.1
GI: 57117473
2626. interleukin-6
[Macropus eugenii]
213 aa protein
AGN92940.1
GI: 511775167
2628. interleukin 6
[Marmota himalayana]
207 aa protein
ABQ82249.1
GI: 148530178
2629. interleukin-6
[Marmota monax]
207 aa protein
CAA74571.1
GI: 2463271
2638. PREDICTED:
interleukin-6-like
[Maylandia zebra]
220 aa protein
XP_004548214.1
GI: 498973754
2662. PREDICTED:
interleukin-6-like,
partial [Meleagris
gallopavo]
125 aa protein
XP_003207178.1
GI: 326921872
2679. PREDICTED:
interleukin-6
[Melopsittacus
undulatus]
287 aa protein
XP_005153103.1
GI: 527270291
2680. interleukin 6
[Meriones
unguiculatus]
186 aa protein
AAS78201.1
GI: 45827246
2695. PREDICTED:
interleukin-6
[Mesocricetus
auratus]
212 aa protein
XP_005087167.1
GI: 524974667
2798. interleukin-6
precursor
[Mus musculus]
211 aa protein
NP_112445.1
GI: 13624311
2993. interleukin 6
[Mustela putorius
furo]
210 aa protein
ABN12937.1
GI: 124491205
3098. Interleukin-6
[Myotis brandtii]
214 aa protein
EPQ11486.1
GI: 521029699
3122. interleukin-6
[Neovison vison]
210 aa protein
ABR18775.1
GI: 148913202
3123. PREDICTED:
interleukin-6
[Nomascus
leucogenys]
212 aa protein
XP_003252690.1
GI: 332207213
3158. interleukin-6,
partial [Numida
meleagris]
48 aa protein
AEI69338.1
GI: 337263392
3177. PREDICTED:
interleukin-6
[Octodon degus]
212 aa protein
XP_004626617.1
GI: 507626208
3190. PREDICTED:
interleukin-6
isoform 1
[Odobenus
rosmarus divergens]
212 aa protein
XP_004397376.1
GI: 472355448
3209. interleukin-6
[Odocoileus
virginianus]
70 aa protein
AAM15962.1
GI: 20257595
3222. interleukin-6
precursor
[Oncorhynchus
mykiss]
219 aa protein
NP_001118129.1
GI: 185134959
3243. PREDICTED:
interleukin-6
[Orcinus orca]
208 aa protein
XP_004263491.1
GI: 465971422
3295. PREDICTED:
interleukin-6-like
isoform 1
[Ornithorhynchus
anatinus]
201 aa protein
XP_001513877.2
GI: 345329348
3309. interleukin-6
precursor
[Oryctolagus
cuniculus]
241 aa protein
NP_001075533.1
GI: 126722667
3333. PREDICTED:
interleukin-6-like
[Oryzias latipes]
224 aa protein
XP_004074037.1
GI: 432882451
3350. PREDICTED:
interleukin-6
[Otolemur garnettii]
278 aa protein
XP_003788649.1
GI: 395831103
3373. interleukin-6
precursor
[Ovis aries]
208 aa protein
NP_001009392.1
GI: 57164389
3436. PREDICTED:
interleukin-6
isoform 3 [Pan
troglodytes]
212 aa protein
XP_518992.2
GI: 114612316
3504. interleukin-6
precursor
[Papio anubis]
212 aa protein
NP_001167007.1
GI: 291084771
3528. interleukin 6
[Paralichthys
olivaceus]
230 aa protein
ABB90401.1
GI: 82780242
3532. interleukin 6
[Passer domesticus]
61 aa protein
ADC79410.1
GI: 288963463
3542. interleukin 6
[Phoca vitulina]
209 aa protein
AAB01430.1
GI: 1161162
3545. interleukin-6,
partial [Phocoena
phocoena]
108 aa protein
AAK19742.1
GI: 13346443
3566. PREDICTED:
interleukin-6
isoform 1
[Pongo abelii]
212 aa protein
XP_002818205.1
GI: 297680890
3599. Interleukin-6,
partial
[Pteropus alecto]
202 aa protein
ELK12578.1
GI: 431908987
3636. interleukin-6
precursor [Rattus
norvegicus]
211 aa protein
NP_036721.1
GI: 7549769
3762. interleukin 6-
like protein
[Retroperitoneal
fibromatosis-
associated
herpesvirus]
209 aa protein
ABX74959.1
GI: 161610411
3764. interleukin-6
homolog
[rhesus monkey
rhadinovirus
H26-95]
207 aa protein
AAC58690.1
GI: 2625047
3767. interleukin-6
[Rousettus
leschenaultii]
207 aa protein
BAH02560.1
GI: 217030543
3782. PREDICTED:
interleukin-6
[Saimiri boliviensis
boliviensis]
212 aa protein
XP_003935160.1
GI: 403287904
3789. interleukin-6
[Saimiri sciureus]
212 aa protein
AAK92044.1
GI: 15213532
3838. PREDICTED:
interleukin-6
[Sorex araneus]
214 aa protein
XP_004604146.1
GI: 505783389
3851. interleukin-6
[Sparus aurata]
225 aa protein
ABY76175.1
GI: 165970321
3860. interleukin 6
[Sus scrofa]
212 aa protein
AAC27127.1
GI: 164515
3891. interleukin-6
precursor
[Sus scrofa]
212 aa protein
NP_999564.1
GI: 47523852
3926. interleukin 6
[Sylvilagus
audubonii]
118 aa protein
AAF86661.1
GI: 9294769
3928. interleukin 6
[Syncerus caffer]
208 aa protein
BAJ11608.1
GI: 300669784
3998. PREDICTED:
interleukin-6
[Taeniopygia
guttata]
180 aa protein
XP_002191320.2
GI: 449493050
4008. interleukin-6
precursor
[Takifugu rubripes]
227 aa protein
NP_001027894.1
GI: 74096421
4042. interleukin-6
[Tetraodon
nigroviridis]
155 aa protein
CAD88198.1
GI: 84619292
4049. PREDICTED:
interleukin-6
[Trichechus
manatus
latirostris]
212 aa protein
XP_004377567.1
GI: 471379540
4062. interleukin-6
[Tupaia
belangeri]
187 aa protein
AGR84924.1
GI: 526304503
4073. Interleukin-6
[Tupaia
chinensis]
171 aa protein
ELW67598.1
GI: 444727091
4089. PREDICTED:
interleukin-6-like
[Tursiops
truncatus]
208 aa protein
XP_004330334.1
GI: 470655819
4103. interleukin 6
[Vulpes vulpes]
207 aa protein
CAF18413.1
GI: 89511614

### Polypeptides of Interest

The present invention includes embodiments comprising IL6⁺-CHO cell lines including one or more polypeptides of interest and/or one or more polynucleotides encoding the polypeptides of interest and methods for making such polypeptides of interest as set forth herein.

A “polynucleotide of interest” is a heterologous polynucleotide, encoding a polypeptide of interest, that is to be expressed by the CHO cell and does not naturally exist in a wild-type CHO cell (e.g., has been added to the CHO cell). For example, in an embodiment of the invention, a polynucleotide of interest is a polynucleotide not encoded by the endogenous genome of a CHO cell. In another embodiment of the invention, a polynucleotide of interest, which may be referred to herein as “heterologous” exists in the endogenous genome of a CHO cell, but wherein an extra copy of the polynucleotide has been added to the CHO cell. In an embodiment of the invention, a polynucleotide of interest encodes an immunoglobulin heavy or light chain. In an embodiment of the invention, the polypeptide of interest is not IL6, such as Chinese hamster IL6 (e.g., not any one or more of the IL6 polynucleotides and/or polypeptides discussed herein) or any one or more of the IL6 pathway members.

In an embodiment of the invention, a polypeptide of interest is an immunoglobulin chain (e.g., from any organism) or therapeutic protein or immunogen (e.g., TSLP (e.g., human, mouse or canine or an immunoglobulin fusion thereof), MK-3475 or an anti-PD1 antibody or antigen-binding fragment thereof, erythropoietin, IL-10, insulin, follicle stimulating hormone, thyrotropin or interferon), e.g., a heavy chain immunoglobulin (e.g., heavy chain variable domain or heavy chain variable domain linked to an immunoglobulin heavy chain constant domain, e.g., gamma-1, gamma-2, gamma-4 or gamma-4) or a light chain immunoglobulin (e.g., light chain variable domain or light chain variable domain linked to an immunoglobulin light chain constant domain, e.g., kappa or lambda).

Polypeptides of interest include immunoglobulin chains (e.g., heavy or light) from antibodies such as MK-3475 (pembrolizumab), dalotuzumab, robatumumab, rituximab, ibritumumab, lambrolizumab, trastuzumab, bevacizumab, cetuximab, panitumumab, ipilimumab, tositumomab, brentuximab, gemtuzumab, alemtuzumab, adecatumumab, labetuzumab, pemtumomab, oregovomab, minretumomab, farletuzumab, etaracizumab, volociximab, cetuximab, nimotuzumab, pertuzumab, mapatumumab, denosumab or sibrotuzumab; or any antigen-binding fragment thereof (e.g., nanobody, Fab, F(ab′)₂, scFv, Fv, diabody, unibody, domain antibody or Fd).

Polynucleotides of interest may, in an embodiment of the invention, be operably linked to a promoter that causes transcription in a CHO cell such as a CMV promoter (e.g., immediate-early cytomegalovirus virus promoter), EF-1alpha promoter, Ubc promoter (human ubiquitin C promoter), SV40 promoter (simian virus 40 promoter) or the PGK promoter (murine phosphoglycerate kinase-1 promoter).

Polypeptides of interest which are immunoglobulins can, in an embodiment of the invention, be used to generate an antibody (e.g., monoclonal, polyclonal, recombinant, fully human, chimeric, humanized, bispecific or anti-idiotypic) or antigen-binding fragment thereof (e.g., nanobody, Fab, F(ab′)₂, scFv, Fv, diabody, unibody, domain antibody or Fd). In such an embodiment of the invention, a method of making an immunoglobulin chain in a CHO cell of the present invention (e.g., as discussed herein) includes the step of combining the immunoglobulin chain the other immunoglobulin chain that is in such an antibody or fragment.

### Chinese Hamster Ovary (CHO) Cells

The present invention includes CHO cells that comprise a polynucleotide encoding polypeptide of interest and a heterologous polynucleotide encoding an IL6 or an IL6 pathway member (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) (e.g., gp130ΔYY). In an embodiment of the invention, the heterologous IL6 or IL6 pathway member encoding polynucleotide is ectopic or is integrated into a chromosome of the CHO cell. In an embodiment of the invention, the polynucleotide of interest is ectopic or is integrated into a chromosome of the CHO cell.

As set forth in the Examples section and the figures, the exposure of CHO cells to IL6 and/or an IL6 pathway member (e.g., gp130ΔYY) has several advantages including:
(i) increasing the number of cells that survive transfection with a vector following selection;
(ii) increasing the quantity of protein of interest expressed from a CHO cell having a polynucleotide encoding the protein of interest (e.g., in fed-batch mode, e.g., following subcloning after transfection with the polynucleotide); and
(iii) increasing the rate of growth (e.g., colony emergence; e.g., in a well such as in a microtiter plate (e.g., 96 well plate)) of CHO cells having a polynucleotide encoding the protein of interest (e.g., following subcloning following transfection with the polynucleotide).

Accordingly, the present invention includes methods for (a) increasing the quantity of protein of interest expressed from a CHO cell having a polynucleotide encoding the polypeptide of interest (e.g., in fed-batch mode, e.g., following subcloning after transfection with the polynucleotide); or for (b) increasing the rate of growth (e.g., colony emergence; e.g., in a well such as in a microtiter plate (e.g., 96 well plate)) of CHO cells having a polynucleotide encoding the protein of interest (e.g., following subcloning following transfection with the polynucleotide) by:
co-expressing IL6 and/or a member of the IL6 pathway with the polypeptide of interest in the CHO cell; or
exposing the CHO cell, having a polynucleotide of interest, to exogenously added IL6 polypeptide.

For example, in an embodiment of the invention, the method includes the step of:
culturing the CHO cell co-expressing IL6 and/or an IL6 pathway member with the polypeptide of interest under conditions favorable to expression of the polypeptide of interest and the IL6 or pathway member; or
culturing the cell expressing the polypeptide of interest in the presence of exogenous IL6 polypeptide (e.g., by adding the exogenous IL6 polypeptide to the culture) under conditions favorable to expression of the polypeptide of interest.

The present invention further includes methods for increasing the number of CHO cells that survive transfection or transformation with a polynucleotide e.g., a polynucleotide of interest, e.g., following selection based on a selectable marker on the polynucleotide, by also transfecting or transformation the cells with a polynucleotide encoding IL6 and/or an IL6 pathway member (e.g., gp130ΔYY) and/or by exposing the cells transfected with the polynucleotide to exogenous IL6 polypeptide.

The present invention provides a method for making a CHO cell of the present invention comprising introducing one or more polynucleotides of interest into the CHO cell and introducing one or more polynucleotides encoding IL6 and/or an IL6 pathway member into the CHO cell, e.g., wherein the polynucleotides are operably linked to a promoter. In an embodiment of the invention, the CHO cell, into which the polynucleotides have been introduced, are subcloned and one or more pure clonal populations of such CHO cells are obtained. In an embodiment of the invention, the cells are subcloned by fluorescence activated cell sorting (FACS), e.g., wherein cells expressing the polypeptide of interest are labeled with an detectably labeled antibody (e.g., phycoerythrin (PE) labeled antibody) that specifically binds the polypeptide of interest and cells labeled with the detectable label are selected and sorted into separate receptacles (e.g., wells of a microtiter plate e.g., with about one cell per receptacle) by a cell sorting machine. In an embodiment of the invention, one or more of the polynucleotides includes a selectable marker (e.g., dihydrofolate reductase (DHFR), glutamine synthetase hygromycin-resistance, puromycin-resistance, or neomycin-resistance) and the CHO cells into which the polynucleotides have been introduced are selected to eliminate or remove cells lacking the polynucleotides.

Compositions comprising an IL6⁺-CHO cell, e.g., in a buffer, culture medium or carrier are part of the present invention as well as methods of making such a composition, e.g., by introducing the cell to the carrier or buffer (e.g., glycerol).

The present invention also provides a composition comprising: (i) exogenous IL6 polypeptide; and (ii) a CHO cell that comprises a polynucleotide of interest, which cell lacks or comprises any IL6 polynucleotide, e.g., wherein both of which are in an aqueous composition, e.g., a liquid growth medium.

CHO cells of the present invention may have certain genetic mutations and/or include additional polynucleotides other than those encoding IL6 and a polynucleotide of interest. For example, in an embodiment of the invention, a CHO cell lacks a functional endogenous DHFR gene and/or glutamine synthase gene. A known method to construct recombinant CHO cell lines is to transfect dihydrofolate reductase negative (dhfr⁻) CHO cell lines with a polynucleotide encoding DHFR and the protein of interest. Methods for making CHO cells of the present invention, in an embodiment of the invention, include the step of selecting CHO cells into which polynucleotides (e.g., encoding IL6 and/or the polypeptide of interest) have been introduced for growth in the absence of glycine, purines, and thymidine. The transfected polynucleotides are then, in an embodiment of the invention, amplified by increasing the concentration of methotrexate (MTX), a competitive inhibitor of DHFR, in the culture medium. During this process the transfected genes are amplified, e.g., several 1000-fold, resulting in an increased production rate for the recombinant protein.

The present invention encompasses CHO cell lines of the present invention wherein the genetic background is that of a CHO K1, DG44 or DUKXB1 cell line. In DG44 cells, both DHFR loci are deleted. DUKXB11, also referred to as DXB11, DUKX, DUKXB1, or DUK-XB11, is derived from CHO K1 cells. The CHO cell line, CHOKISV, is a variant of the cell line CHO-KI that has been adapted to growth in suspension and protein-free medium. The present invention encompasses IL6⁺-CHO cells and methods of using and making CHO cells (e.g., IL6⁺-CHO cells) wherein the cells have a CHOKISV, CHO K1, DG44 or DUKXB1 cell genetic background (as discussed herein). The present invention includes the CHO cells discussed herein and their methods of use and methods of making the same having the genetic background of any CHO cell, including those specifically discussed herein.

An IL6⁺-CHO cell line of the present invention, for example, with a DG44 “genetic background” is, genetically, essentially identical to DG44 but for including the polynucleotides encoding IL6 and/or an IL6 pathway member and the polypeptide of interest (and any other genetic modifications a user may introduce) and can be created, for example, by introducing the polynucleotide of interest and the polynucleotide encoding the IL6 into a DG44 cell line.

In an embodiment of the invention, a CHO cell of the present invention lacks a functional FUT8 polypeptide (e.g., wherein two chromosomal copies of CHO FUT8 have been mutated or expression has been inhibited, e.g., by use of RNA interference or anti-sense RNA or DNA). The present invention includes such cells and methods of using and making the same.

In an embodiment of the invention, a CHO cell of the present invention lacks a functional glutamine synthase (GS) polypeptide (e.g., wherein two chromosomal copies of CHO GS have been mutated or expression has been inhibited, e.g., by use of RNA interference or anti-sense RNA or DNA). The present invention includes such cells and methods of using and making the same.

The present invention includes a method for growing an IL6⁺-CHO cell of the present invention comprising introducing the cell to a growth medium and culturing the cell under conditions favorable to such growth, e.g., in the presence of exogenously added IL6 polypeptide. In embodiments wherein the CHO cells have a polynucleotide encoding IL6 or a polypeptide of interest, in a vector comprising a selectable marker, the method of growing the cells optionally includes the step of culturing the cell under conditions wherein growth of cells that lack the marker is inhibited, e.g., wherein the selectable marker is dihydrofolate reductase (DHFR), growing the cell in the presence of methotrexate; wherein the selectable marker is glutamine synthetase, growing the cells in the absence of glutamine and/or in the presence of methionine sulphoxamine (MSX; e.g., at about 25 micromolar); wherein the selectable marker is hygromycin-resistance, growing the cells in the presence of hygromycin; wherein the selectable marker is puromycin-resistance, growing the cells in the presence of puromycin; or, wherein the selectable marker is neomycin-resistance growing the cells in the presence of geneticin (G418).

### Expression

The present invention provides methods for expressing a polypeptide of interest in an IL6⁺-CHO cell. Such a method comprises: culturing an IL6⁺-CHO cell under conditions wherein the IL6 and/or IL6 pathway member (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) is expressed and secreted and the polynucleotide of interest is expressed (and, optionally secreted). Optionally, the method includes the steps for making the IL6⁺-CHO cells which are discussed herein. Optionally, the cell culture is supplemented with exogenously added IL6 polypeptide (e.g., about 100 ng/ml; e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta)

The present invention also provides a method for expressing a polypeptide of interest in a CHO cell which comprises a polynucleotide encoding the polypeptide of interest, but which does not include a IL6 polynucleotide. Such a method comprises culturing the cell in the presence of exogenously added IL6 polypeptide (e.g., comprising the step of adding exogenous IL6 polypeptide to the culture medium) under conditions wherein the polynucleotide of interest is expressed.

As discussed above, expression of a polynucleotide encoding a polypeptide of interest or IL6 in a CHO cell may be driven by any of several promoters known in the art such as a CMV promoter (e.g., immediate-early cytomegalovirus virus promoter), EF-1alpha promoter, Ubc promoter (human ubiquitin C promoter), SV40 promoter (simian virus 40 promoter) or the PGK promoter (murine phosphoglycerate kinase-1 promoter).

In embodiments of the invention, wherein the CHO cell has a polynucleotide encoding IL6 and/or an IL6 pathway member (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta) or a polypeptide of interest in a vector comprising a selectable marker (e.g., dihydrofolate reductase (DHFR), glutamine synthetase hygromycin-resistance, puromycin-resistance, or neomycin-resistance), the method of expressing the polypeptide of interest optionally includes the steps of: wherein the selectable marker is dihydrofolate reductase (DHFR), growing the cell in the presence of methotrexate; wherein the selectable marker is glutamine synthetase, growing the cells in the absence of glutamine and/or in the presence of methionine sulphoxamine (MSX); wherein the selectable marker is hygromycin-resistance, growing the cells in the presence of hygromycin; wherein the selectable marker is puromycin-resistance, growing the cells in the presence of puromycin; or, wherein the selectable marker is neomycin-resistance growing the cells in the presence of geneticin (G418).

Methods for culturing CHO cells are known in the art. The present invention includes, e.g., growth of CHO cells for expression of a polypeptide of interest by the fed-batch method. In a fed-batch process, a basal medium supports initial growth and production, and a controlled addition of feed medium to the cell culture prevents depletion of nutrients and sustains the production phase of the cells and the polypeptide of interest.

Cell lines of the present invention can be cultured under conditions favorable to the expression of the polypeptide of interest using culture mediums that are commonly available in the art. Examples of available media include BM-1, BM-2 or BM-3 medium, Roswell Park Memorial Institute (RPMI) 1640 medium, L-15 medium, Dulbecco's modified Eagle's medium (DMEM), Eagle's minimal essential medium (MEM), Ham's F12 medium. Mediums include, for example, animal-component free, serum-free and/or protein-free media. For example, such a medium may contain inorganic salts, buffers such as (HEPES and/or sodium bicarbonate buffers), essential and non-essential amino acids, vitamins, recombinant human insulin, plant hydrolysates, trace elements, and/or surfactants.

Additional substances that may be added to a CHO cell culture medium include, e.g., transferrin, insulin, bovine serum albumin, pluronic F68, polyethylene glycol, glucose, cholesterol, lipids (e.g., oleic acid, linoleic acid or phospholipids), vitamins (e.g., A, D, E, K, biotin, choline, folic acid, inositol, niacinamide, pantothenic acid, pyridoxine, riboflavin or thiamine, ascorbic acid, nicotinamide or choline), trace elements (e.g., copper, iron, manganese, selenium or zinc).

Cell lines of the present invention may be adapted for growth in serum-free conditions using methods known in the art, for example, stepwise decreasing the presence of serum or protein in the culture medium over time. For example, protein-free adaptation may be obtained by stepwise decreasing the presence of protein in the medium over time in an already serum-free medium. The present invention encompasses methods discussed herein wherein the CHO cell lines of the present invention are obtained or used wherein such cell lines have been adapted for growth in serum-free and/or protein-free media. Serum-free and/or protein-free adapted CHO cells (as discussed herein) are part of the present invention as are methods of using such CHO cells (as discussed herein). Methods of making and using the CHO cells of the present invention (as discussed herein), wherein the methods include the step of adapting the cells for serum-free and/or protein-free growth, are part of the present invention.

Polypeptides of interest that are expressed in a CHO cell of the present invention may be isolated from the cells and/or the culture medium, e.g., and purified, e.g., chromatographically, e.g., using centrifugation, depth filtration, cation exchange, ion exchange, hydrophobic interaction chromatography and/or size exclusion chromatography. Methods of using the CHO cells (as discussed herein), e.g., for protein expression, that include steps for such purification are part of the present invention.

In an embodiment of the invention, a CHO cell is cultured at a pH of about 6 to about 7.5 (e.g., 6.0, 6.25, 6.5, 7.0, 7.25, 7.5).

In an embodiment of the invention, a CHO cell is cultured in a medium having a percentage of dissolved O₂ of about 30% to about 50% (e.g., 30%, 35%, 40%, 45% or 50%).

In an embodiment of the invention, a CHO cell is cultured in a medium at about 37° C.

IL6⁺-CHO cell growth can be performed in any of several systems. For example, cell culture growth can be done in a simple flask, e.g., a glass shake flask. Other systems include tank bioreactors, bag bioreactors and disposable bioreactors. A tank bioreactor includes, typically, a metal vessel (e.g., a stainless steel jacketed vessel) in which cells are grown in a liquid medium. Tank bioreactors can be used for a wide range of culture volumes (e.g., 100 I, 150 I, 10000 I, 15000 I). Tank bioreactors often have additional features for controlling cell growth conditions, including means for temperature control, medium agitation, controlling sparge gas concentrations, controlling pH, controlling O₂ concentration, removing samples from the medium, reactor weight indication and control, cleaning hardware, sterilizing the hardware, piping or tubing to deliver all services, adding media, control pH, control solutions, and control gases, pumping sterile fluids into the growth vessel and, supervisory control and a data acquisition. Classifications of tank bioreactor include stirred tank reactors wherein mechanical stirrers (e.g., impellers) are used to mix the reactor to distribute heat and materials (such as oxygen and substrates). Bubble column reactors are tall reactors which use air alone to mix the contents. Air lift reactors are similar to bubble column reactors, but differ by the fact that they contain a draft tube. The draft tube is typically an inner tube which improves circulation and oxygen transfer and equalizes shear forces in the reactor. In fluidized bed reactors, cells are “immobilized” on small particles which move with the fluid. The small particles create a large surface area for cells to stick to and enable a high rate of transfer of oxygen and nutrients to the cells. In packed bed reactors cells are immobilized on large particles. These particles do not move with the liquid. Packed bed reactors are simple to construct and operate but can suffer from blockages and from poor oxygen transfer. A disposable bioreactor is a disposable, one-time use bioreactor. Often, disposable bioreactors possess features similar to non-disposable bioreactors (e.g., agitation system, sparge, probes, ports, etc.). Any method of making or using a IL6⁺-CHO cell of the present invention (e.g., for protein expression) can include, in an embodiment of the invention, growth of the cell in a system as discussed herein.

The present invention includes not only individual isolated IL6⁺-CHO cells but also master cell banks (MCB) and working cell banks (WCB), e.g., comprising the IL6⁺-CHO cells. Typically, when a cell line is to be used over many manufacturing cycles, a two-tiered cell banking system consisting of a master cell bank or master seed bank and a working cell bank can be established. A cell line is generally established from a single host cell clone and this cell line is used to make-up the MCB. Generally, this MCB must be characterized and extensively tested for contaminants such as bacteria, fungi, viruses and mycoplasma. A sample of cells from the MCB can be expanded to form the WCB, which is characterized for cell viability prior to use in a manufacturing process. The cells in a MCB or WCB can be stored in vials, for example, at low temperature (e.g., 0° C. or lower, −20° C. or −80° C., or in liquid nitrogen, e.g., at −110° C. to −180° C.). Typically, the working cell bank includes cells from one vial of the master bank which have been grown for several passages before storage. In general, when future cells are needed, they are taken from the working cell bank; whereas, the master cell bank is used only when necessary, ensuring a stock of cells with a low passage number to avoid genetic variation within the cell culture. Any of the methods of using the IL6⁺-CHO cells of the present invention (e.g., for protein expression as discussed herein) can, in an embodiment of the invention, include the step of obtaining the cell from cell from a master cell bank and/or working cell bank before use (e.g., comprising the step of thawing the cell from the cold MCB or WCB storage conditions). The present invention also includes methods of making a master cell bank and/or working cell bank from a CHO cell of the present invention, e.g., comprising placing the cell in a medium suitable for cold storage and storing the cell at a low temperature (e.g., 0° C. or lower, −20° C. or −80° C., or in liquid nitrogen, e.g., at −110° C. to −180° C.).

### Kits

The present invention also includes kits comprising the a CHO cell (e.g., IL6⁺-CHO and/or a CHO cell that lacks a polynucleotide encoding a heterologous IL6), instructions for use and optionally, exogenous IL6 polypeptide (e.g., mammal, human, hamster such as Chinese hamster (e.g., Cricetulus griseus), rat such as Rattus norvegicus, mouse such as Mus musculus, chimp such as Pan troglodytes, gorilla such as Gorilla gorilla gorilla or monkey such as Macaca mulatta). Other kit components may include transfection reagents for introducing a polynucleotide of interest into the CHO cell (e.g., the polynucleotide of interest itself or a polynucleotide encoding IL6 and/or an IL6 pathway member) and a culture medium or culture medium components. Methods for making such kits including the step of combining the components of the kit are part of the present invention.

### EXAMPLES

### Example 1

### Generation of CHO Cell Line

Human IL6 was obtained from Sigma Aldrich. Human and Chinese hamster IL6 amino acid sequences were obtained from the National Center for Biotechnology Information and codon optimized and synthesized.

Human or CHO IL6 was cloned into a vector carrying a canine protein fused to mouse Fc (canP-mIgG) to yield pCanP-mIgG+hIL6 and pCanP-mIgG+CHOIL6, respectively. In experiments testing exogenous human IL6, CHOK1SV cells were transfected with Pvul-linearized pCanP-mIgG by electroporation with the Gene Pulser Xcell electroporator unit (BioRad). Transfected cells were recovered in the presence or absence of 100 ng/ml human IL6 for 24 hours, then selected (based on the selectable marker in the transfected DNA vector backbone) in the presence or absence of 100 ng/ml human IL6 for 14 days.

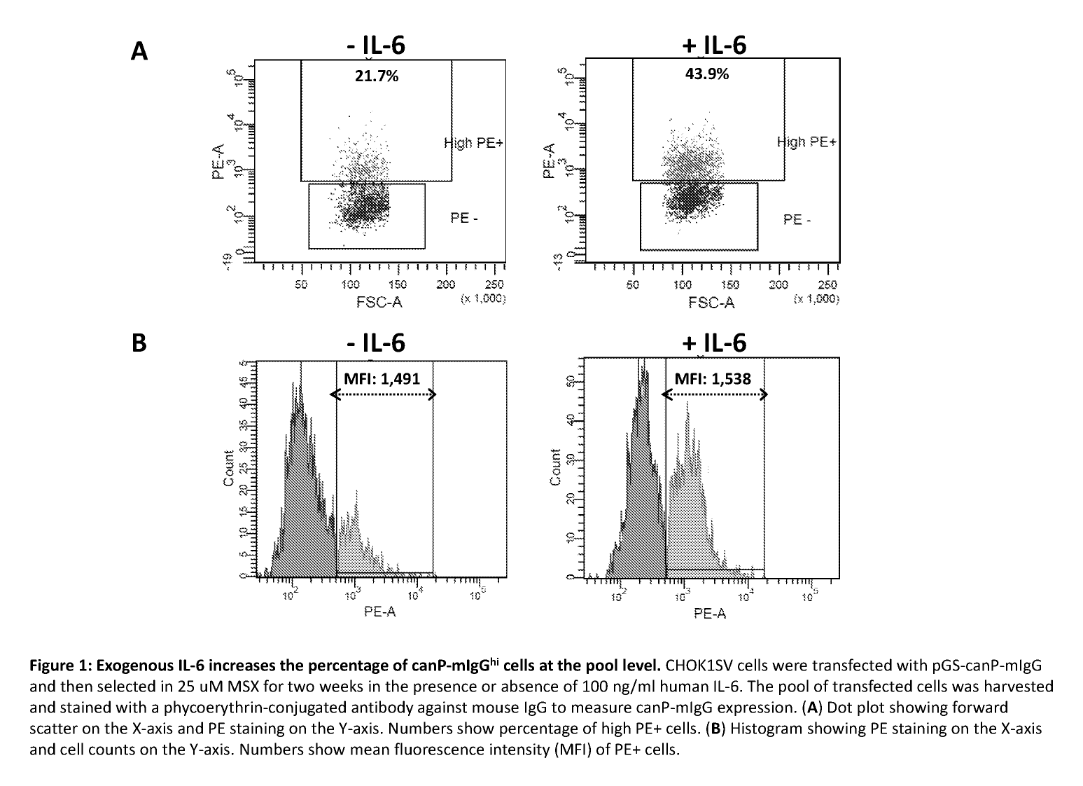

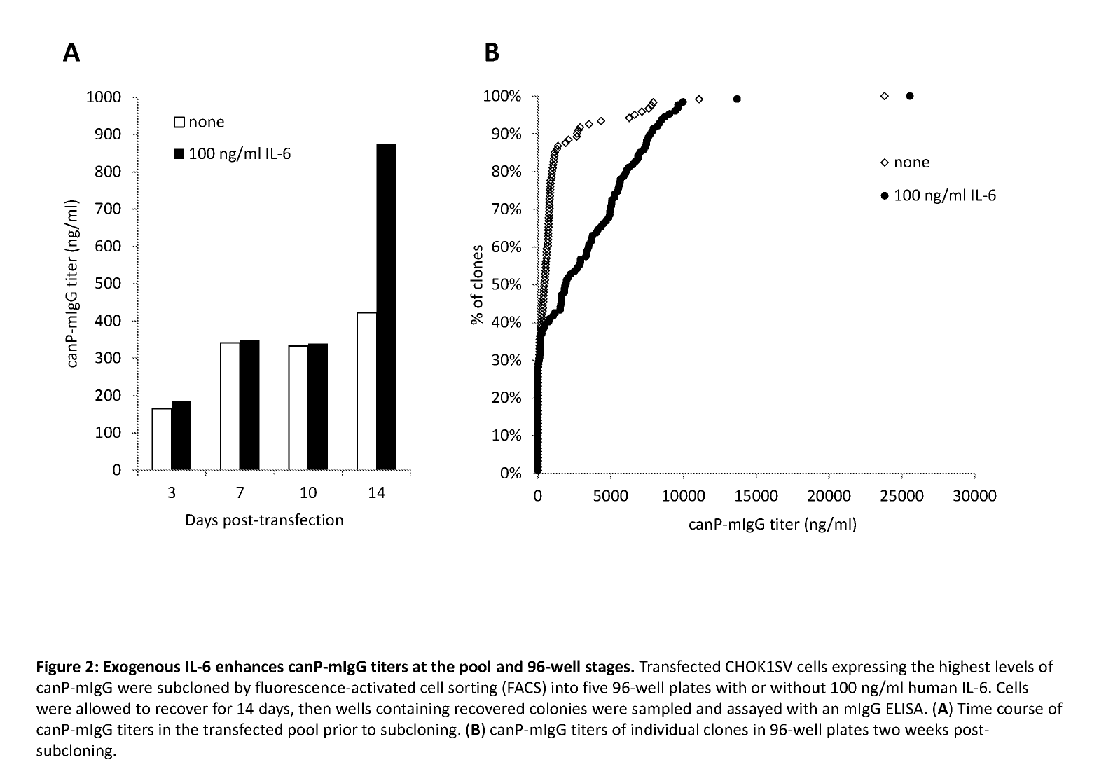

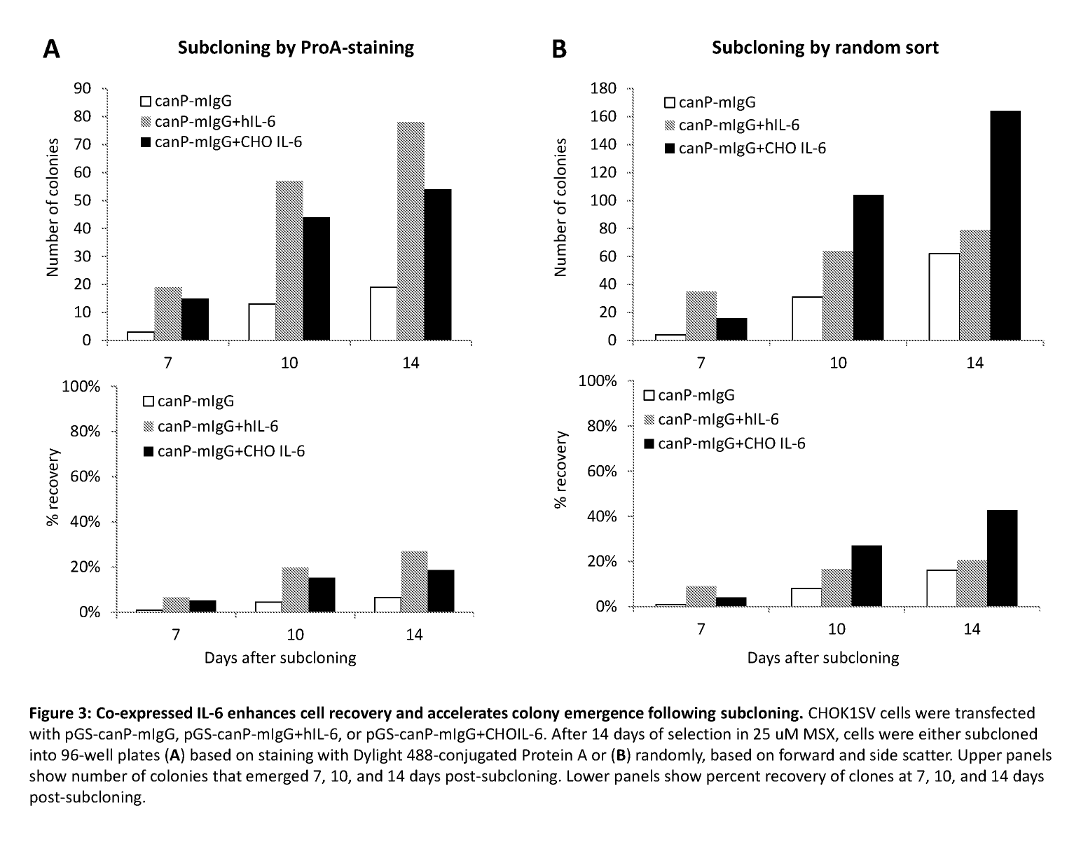

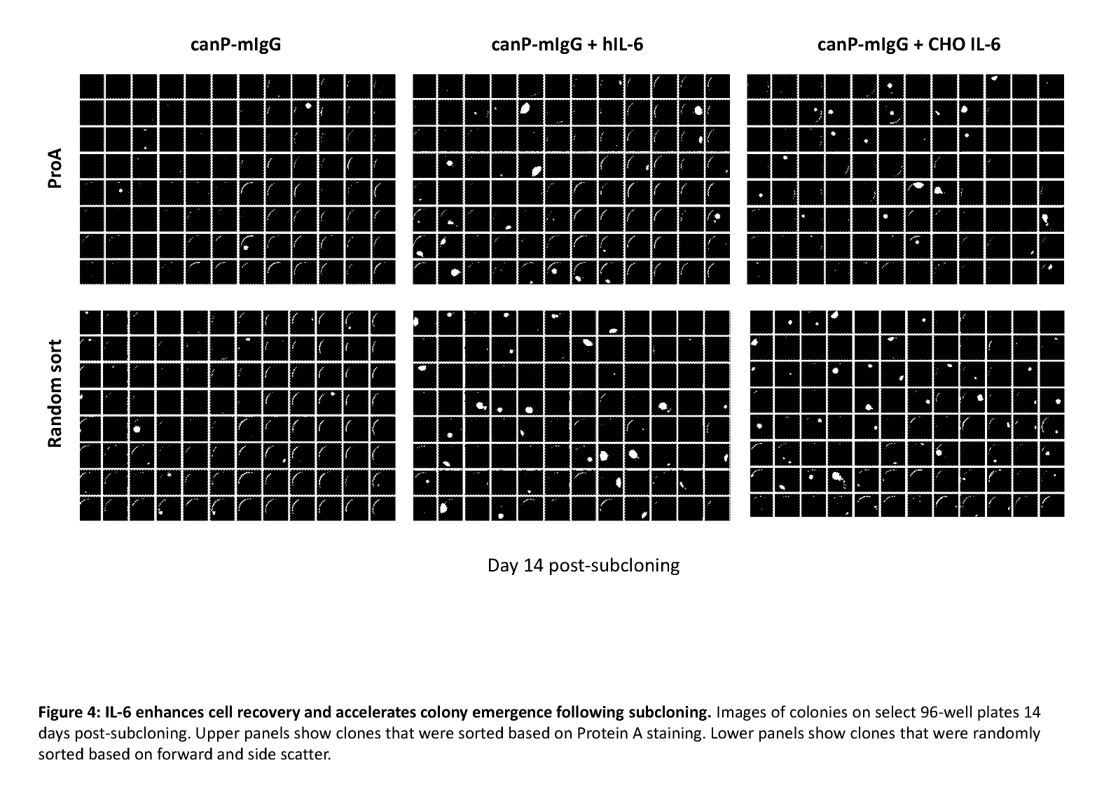

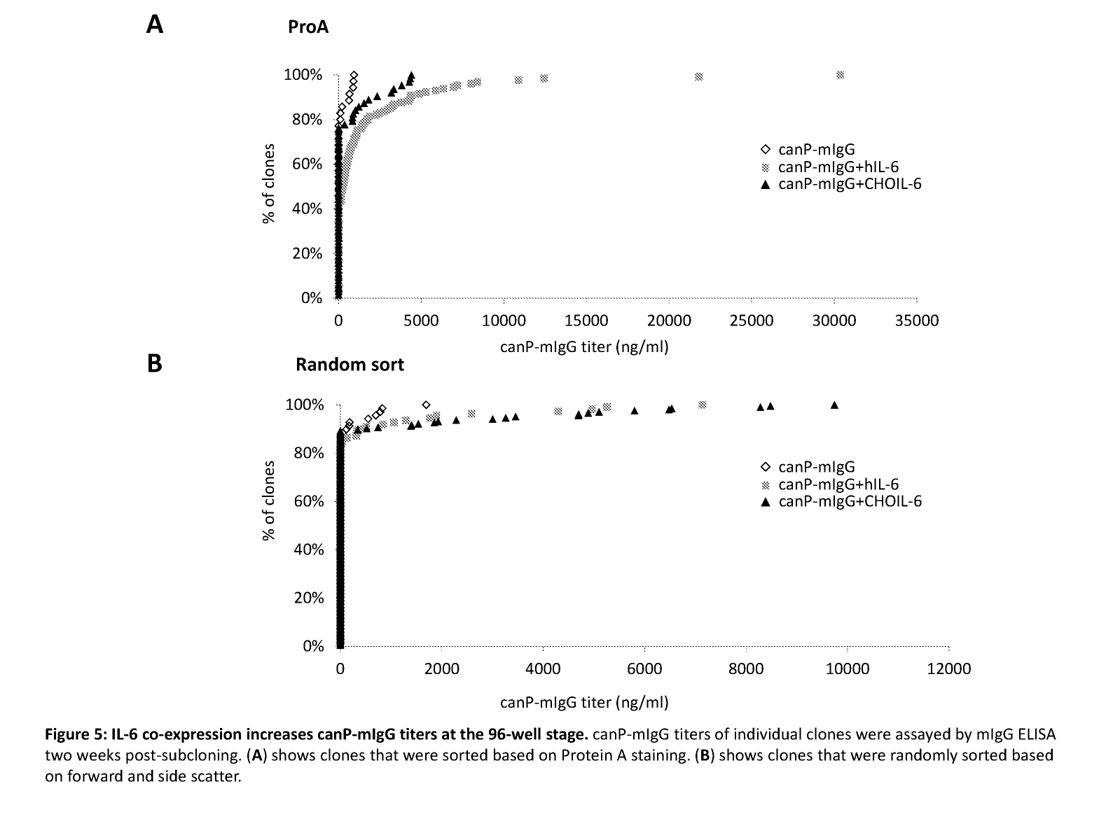

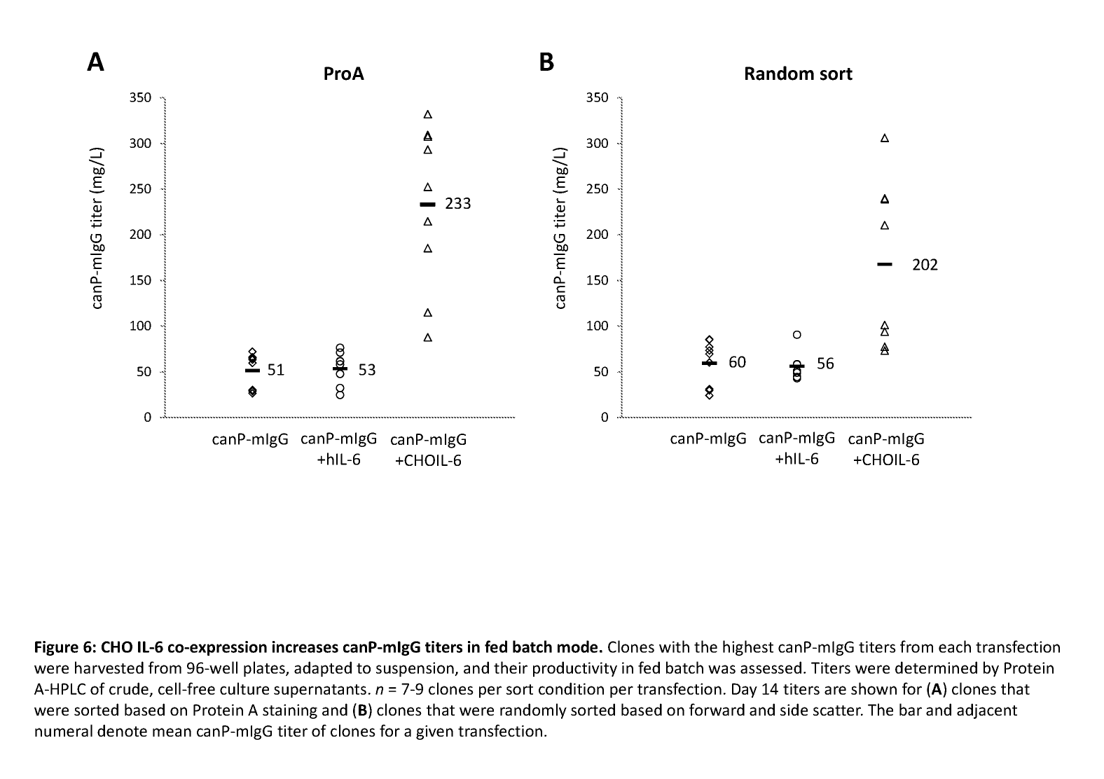

The pool of transfected cells was harvested and stained with a phycoerythrin-conjugated antibody against mouse IgG (Jackson ImmunoResearch) to measure canP-mIgG expression (FIG. 1). Cells expressing the highest levels of canP-mIgG were subcloned by fluorescence-activated cell sorting (FACS) into five 96-well plates with or without 100 ng/ml human IL6. Cells were allowed to recover for 14 days, then wells containing recovered colonies were sampled and assayed with an mIgG ELISA (Bethyl Labs, FIG. 2). In experiments testing co-expressed human or CHO IL6, CHOK1SV cells were transfected with Pvul-linearized pCanP-mIgG, pCanP-mIgG+hIL6, or pMIgG +CHOIL6 by electroporation, as described above. Transfected cells were again selected based on the selectable marker for 14 days. The pool of transfected cells was harvested, and a portion was stained with Dylight 488-conjugated Protein A (Rockland Immunochemicals) to measure canP-mIgG expression. Cells expressing the highest levels of canP-mIgG were subcloned by FACS into five 96-well plates. Another portion of cells was subcloned by random sorting into five 96-well plates. Cells were allowed to recover for 14 days, then wells containing recovered colonies were sampled and assayed with an mIgG ELISA (Bethyl Labs, FIGS. 3-5). In addition, cells transfected with pCanPmIgG+hIL6 were assayed with an hIL6 ELISA (eBioscience). Clones with the highest canP-mIgG titers from each transfection were harvested, adapted to suspension, and their productivity in a 14-day fed batch process was assessed (FIG. 6).

### Example 2

### Characterization of CHO Cells Lines Exposed to IL-6

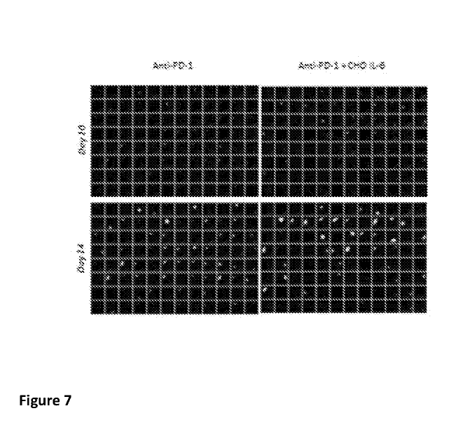

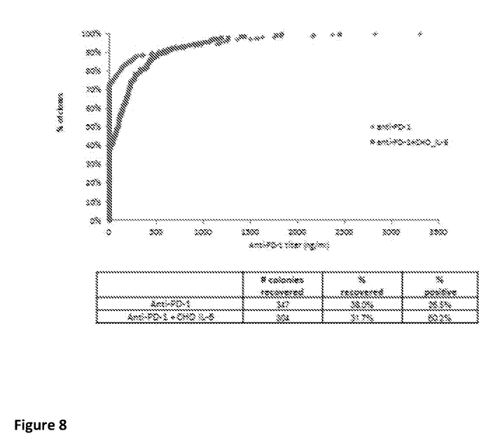

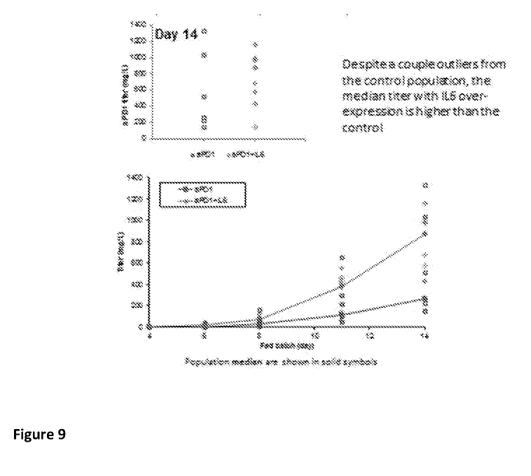

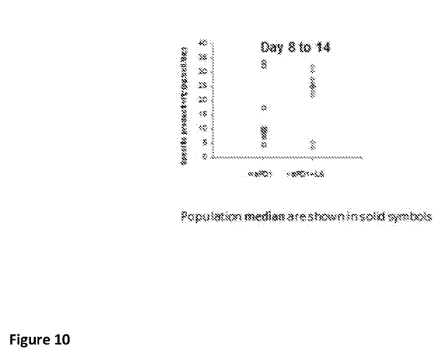

In this study, we found that supplementation with IL-6 or co-expressing IL-6 during cell line development process promoted cell growth and recombinant protein productivity. We demonstrated that overexpression of CHO IL-6 improved cell growth (FIG. 7) and antibody production (FIG. 8) during cloning stage as well as under fed-batch conditions (FIGS. 9-10) of MK-3475 (anti-PD1 antibody) production cell lines.

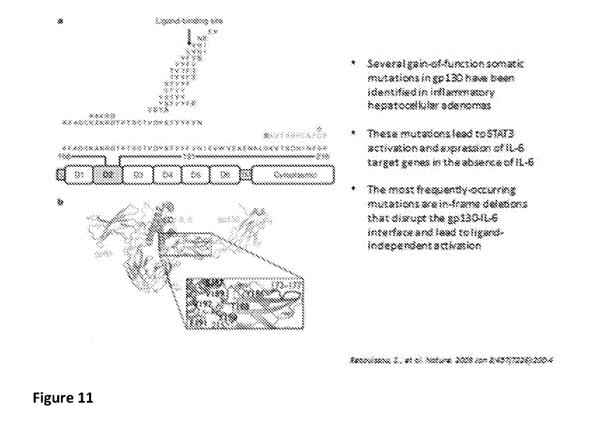

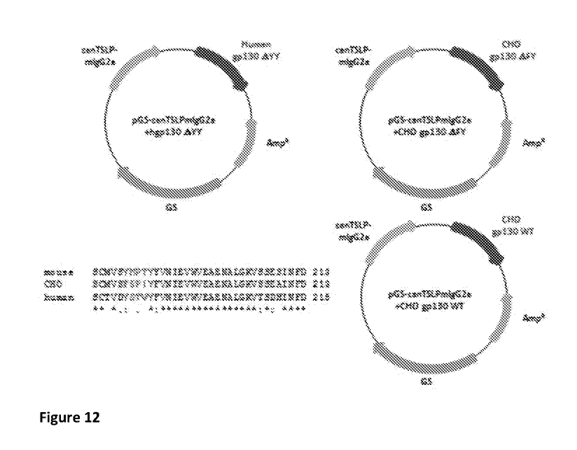

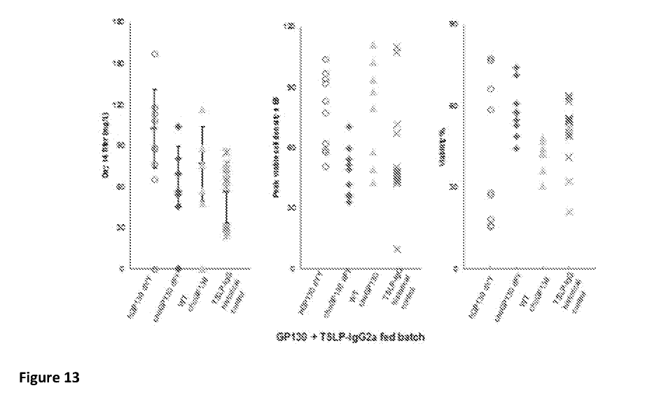

On the other hand, we found the overexpression of a constitutively active IL-6 receptor variant (human gp130ΔYY; Rebouissou et al. Nature 457: 200-204 (2009)) achieved a similar effect as co-expressing IL-6. Several gain-of-function human gp130 variants are listed in FIG. 11. We chose to test the ΔYY in-frame deletion variant (FIG. 12) which is a well-studied mutant leading to IL-6 independent activation of STAT3 signaling pathway. Overexpression of human gp130ΔYY significantly improved productivity of the TSLP-IgG production cell lines (FIG. 13). Thus, we propose that cytokines signaling pathway engineering can be applied to the cell line development process to reduce clone selection timelines, improve cell line productivity, and facilitate the identification of highly-productive clones.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

1. An isolated Chinese hamster ovary cell comprising a heterologous polynucleotide encoding a polypeptide of interest:
(a) further comprising a heterologous polynucleotide encoding IL6 and/or an IL6 pathway member; and/or
(b) in the presence of exogenous IL6 polypeptide.

2. The cell of claim 1 wherein the cell comprises the polynucleotide encoding a polypeptide of interest and is genetically heterozygous or homozygous for the gp130ΔYY or gp130ΔFY mutant allele.

3. The cell of claim 1 wherein the IL6 is Chinese hamster IL6.

4. The cell of claim 1 wherein the polypeptide of interest is an immunoglobulin.

5. The cell of claim 1 which:
(a) lacks a functional FUT8 polypeptide;
(b) lacks a functional glutamine synthase polypeptide; and/or
(c) lacks a functional DHFR polypeptide.

6. The cell of claim 1 which has a CHO K1, DG44 or DUKXB1 genetic background.

7. A composition comprising the cell of claim 1 in a culture medium, buffer or carrier.

8. A method for increasing the quantity of a protein of interest expressed from a Chinese hamster ovary cell having a polynucleotide encoding the protein of interest; or, for increasing the rate of growth of a Chinese hamster ovary cell having a polynucleotide encoding the protein of interest; comprising: co-expressing IL6 and/or an IL6 pathway member with the polypeptide of interest in the Chinese hamster ovary cell; and/or, exposing a Chinese hamster ovary cell having a polynucleotide encoding the protein of interest to exogenous IL6 polypeptide.

9. A method for increasing the number of Chinese hamster ovary cells that survive transfection with a polynucleotide comprising: transfecting the cells with a polynucleotide encoding IL6 and/or an IL6 pathway member and/or exposing the cells transfected with the polynucleotide to exogenous IL6 polypeptide.

10. A method for making the cell of claim 1 comprising introducing a polynucleotide encoding a polypeptide of interest and a polynucleotide encoding IL6 polypeptide and/or an IL6 pathway member polypeptide into a Chinese hamster ovary cell.

11. A method for making a polypeptide of interest comprising culturing the cell of claim 1 under conditions wherein the polypeptide of interest and the IL6 polypeptide and/or an IL6 pathway member polypeptide are expressed.

12. The method of claim 11 that comprises the steps of:
(a) introducing into the Chinese hamster ovary cell:
(i) a polynucleotide encoding the polypeptide of interest, and
(ii) a polynucleotide encoding IL6 polypeptide and/or an IL6 pathway member polypeptide; and
(b) culturing the cell under conditions wherein the polypeptide of interest and the IL6 polypeptide and/or an IL6 pathway member polypeptide are expressed.

13. The method of claim 11 that comprise the steps of:
(a) introducing a polynucleotide encoding a polypeptide of interest into the Chinese hamster ovary cell;
(b) introducing the cell into a growth medium;
(c) adding exogenous IL6 polypeptide to the growth medium; and
(d) culturing the cell under conditions wherein the polypeptide of interest and the IL6 and/or IL6 pathway member are expressed.

14. The method of claim 8 wherein the IL6 pathway member is gp130ΔYY or gp130ΔFY mutant.


SEQUENCE LISTING (8 sequences)

SEQ ID NO: 1 - DNA, 639 bp, Homo sapiens
  CDS (1)..(639)
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - protein, 212 aa, Cricetulus griseus
MKFLSARDFHPLAFLGLMLAVATALPTSQVRRGDFTEDTTPNRPVYTTSQQVGGLVTHVLREIFELRKELCNNNPDCMNYDDALLENNLELPVIQRNDGCYQTGYNWEICLLKITSGLLDYQIYLEFVTNNVQDNKKDKARVIQSTTKTLSQIFKQEVKDPDKIVMPSPTSKAILIEKLESQKQWPRTKTIELILKALEEFLKVTMRSTRQN

SEQ ID NO: 4 - protein, 211 aa, Rattus norvegicus
MKFLSARDFQPVAFLGLMLLTATAFPTSQVRRGDFTEDTTHNRPVYTTSQVGGLITYVLREILEMRKELCNGNSDCMNSDDALSENNLKLPEIQRNDGCFQTGYNQEICLLKICSGLLEFRFYLEFVKNNLQDNKKDKARVIQSNTETLVHIFKQEIKDSYKIVLPTPTSNALLMEKLESQKEWLRTKTIQLILKALEEFLKVTMRSTRQT

SEQ ID NO: 5 - protein, 194 aa, Mus musculus
MLVTTTAFPTSQVRRGDFTEDTTHNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 6 - protein, 212 aa, Pan troglodytes
MNSVSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 7 - protein, 212 aa, Gorilla gorilla gorilla
MNSVSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKVGNLDVITTPDPTTNASLLTKLQAQNQWLQDMTTHLILRSFKEFLQSSLRALRQM

SEQ ID NO: 8 - protein, 212 aa, Macaca mulatta
MNSVSTSAFGPVAFSLGLLLVLPAAFPAPVLPGEDSKDVAAPHSQPLTSSERIDKHIRYILDGISALRKETCNRSNMCESSKEALAENNLNLPKMAEKDGCFQSGFNEDTCLVKIITGLLEFEVYLEYLQNRFESSEEQARAVQMSTKVLIQFLQKKAKNLDAITTPEPTTNASLLTKLQAQNQWLQDMTTHLILRSFKEFLQSSLRALRQM